(19) United States
(12) Patent Application Publication
(10) Pub. No.: US 2026/0096830 A1
(43) Pub. Date: Apr. 9, 2026

(54) UNIVERSAL SCORING CATHETER

(71) Applicant: BARD PERIPHERAL VASCULAR, INC., Franklin Lakes, NJ (US)

(72) Inventors: Andrzej J. Chanduszko, Chandler, AZ (US); Matthew R. Casiraro, Tempe, AZ (US)

(21) Appl. No.: 19/110,420

(22) PCT Filed: Sep. 8, 2022
(86) PCT No.: PCT/US2022/042838

§ 371 Date: Mar. 10, 2025

(51) Int. Cl. A61B 17/3207; A61B 17/00

(52) U.S. Cl. CPC A61B 17/320725; A61B 2017/00557; A61B 2017/00778

(57) ABSTRACT

An apparatus for performing a medical procedure on a structure, such as a lesion, in a vessel. The apparatus includes a catheter shaft having a frangible portion with a scoring element adapted for scoring the lesion when the frangible portion is broken. An expandable element, such as an inflatable balloon, may be provided for breaking the frangible portion, which then allows the scoring element to move radially outwardly toward the structure or lesion in the vessel. Related methods are also disclosed.

### BACKGROUND

Dilatation catheters with expandable elements or balloons are often used to treat lesions in vessels. However, difficulties are encountered in navigating tortuous anatomy and safely crossing very tight lesions. Moreover, some lesions are difficult to dilate using just a balloon, and require the application of a focused force, such as provided by a scoring wire to dilate the lesion.

Current balloons with scoring wires are typically selected based on the length relative to the lesion, which may require an increased product inventory. The scoring wire is also typically attached directly to the balloon during manufacturing. This makes the arrangement fully dependent on a particular balloon size (length or diameter) to achieve results, and thus lacks universal applicability among a variety of balloons, such as those of different distributors. This also inhibits the ability to customize the scoring elements for a particular balloon catheter.

Accordingly, a need is identified for a catheter with enhanced scoring capabilities that overcomes the foregoing limitations, and possibly others that have yet to be discovered. The scoring capability would be easy and inexpensive to implement using a variety of existing balloon catheters with different dimensions without limitation, and thus could have more universal applicability than currently possible using existing approaches where a scoring element in the form of a wire that extends along a balloon.

### SUMMARY

An object of the disclosure is to provide a catheter with enhanced scoring capabilities. In particular, the catheter would afford scoring capability in an easy and inexpensive manner using a variety of existing balloon catheters with different dimensions, and thus in a sense would have universal applicability. By using a catheter shaft for receiving the expandable element including one or more perforated portions for carrying one or more scoring elements, the risk of accidental or unintentional deployment would be minimized or eliminated. The scoring capabilities could also be customized independent of the balloon.

According to one aspect of the disclosure, an apparatus for scoring a lesion in a vessel is provided. The apparatus includes a catheter shaft comprising a frangible portion. The frangible portion includes a scoring element adapted for advancing radially outwardly for scoring the lesion when the frangible portion is broken.

In one embodiment, the frangible portion comprises perforations arranged on either side of the scoring element, which may include one or more beads, blades, serrations, or a combination thereof. The catheter shaft may comprise a plurality of frangible portions circumferentially spaced around the catheter shaft, each associated with a scoring element. Each frangible portion may comprise longitudinal perforations arranged on either side of the scoring element.

In one particular example, the scoring element has a length in a longitudinal direction less than a length of the frangible portion. The frangible portion may comprise a radially depressed portion of the catheter shaft.

An expandable element may be provided within the shaft for breaking the frangible portion. The expandable element may comprise a balloon.

According to a further aspect of the disclosure, an apparatus for scoring a lesion in a vessel is provided. The apparatus comprises a catheter shaft including an expandable portion adapted for scoring the lesion. The catheter shaft includes a plurality of perforations forming the expandable portion.

In one embodiment, the expandable portion of the catheter shaft includes a scoring element, and the plurality of perforations comprise perforations located on either side of the scoring element. The scoring element may comprise one or more beads, blades, serrations, or a combination thereof.

The expandable portion may optionally comprise a depressed portion of the catheter shaft. The catheter shaft may include a plurality of scoring elements, and the plurality of perforations comprise perforations located on either side of the depressed portion.

A balloon catheter may be adapted to be inserted into the catheter shaft. Expansion of the balloon thus serves to deploy the expandable portion, such as by breaking or tearing perforations connecting it with the catheter shaft. The balloon may have a length equal to or less than a length of the expandable portion.

In accordance with a further aspect of the disclosure, an apparatus for scoring a lesion in a vessel is provided. The apparatus includes a catheter shaft with a depressed portion having a scoring element adapted for scoring the lesion when the depressed portion is urged radially outwardly to a deployed position. The depressed portion may further comprise a frangible portion of the catheter. An expandable element may be provided within the shaft for breaking the frangible portion, such as a balloon associated with a balloon catheter.

This disclosure also relates to a method for scoring a lesion in a vessel in connection with an expandable element. The method comprises breaking apart a catheter shaft to cause a scoring portion thereon to assume a deployed position for scoring the lesion.

In one embodiment, the breaking apart step comprises expanding an expandable balloon within the catheter shaft. In a more specific embodiment, the breaking apart step comprises tearing perforations formed in the catheter shaft on either side of a scoring element.

Also an aspect of this disclosure is a method for scoring a lesion in a vessel in connection with an expandable element. The method comprises expanding a portion of catheter shaft from a depressed position to cause a scoring element thereon to assume a deployed position for scoring the lesion. In one embodiment, the expanding step comprises breaking a frangible portion of the catheter, which may involve tearing perforations formed in the catheter shaft on either side of the scoring element.

### BRIEF DESCRIPTION OF THE DRAWINGS

The above and further advantages of the disclosure may be better understood by referring to the following description in conjunction with the accompanying drawings in which:

FIG. 1 is a perspective view of a balloon catheter;

FIG. 1A is a cross-sectional view taken along line 1A-1A of FIG. 1;

FIG. 2 is a side-view of a scoring catheter shaft according to one aspect of the disclosure;

FIG. 2A is a cross-sectional view taken along line 2A-2A of FIG. 2;

FIG. 3 is a side view of an expanded version of the scoring catheter shaft of FIG. 2;

FIG. 4 is an alternative embodiment; and

FIG. 5 is a cross-sectional view of a balloon in a folded condition in the scoring catheter shaft prior to deployment.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the invention(s) of the present disclosure.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

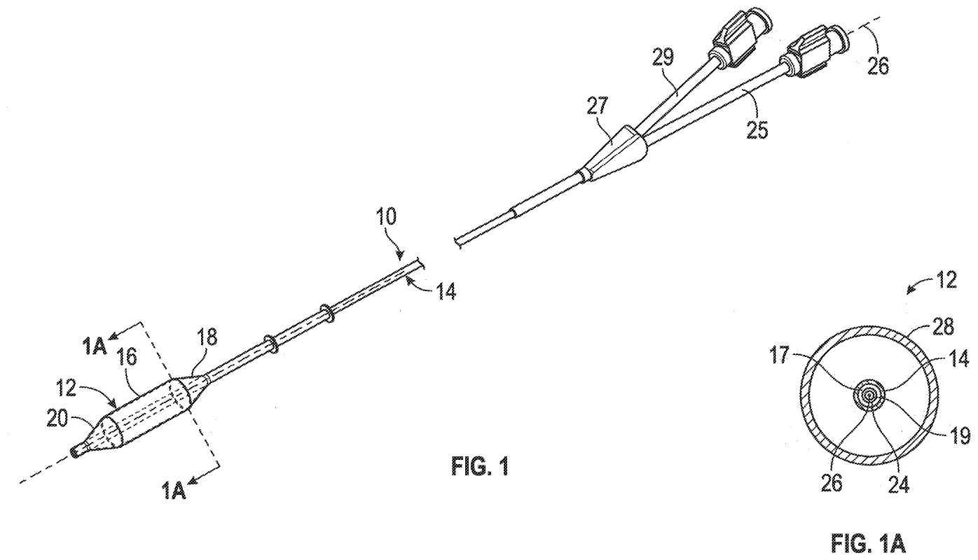

With reference to FIG. 1, provided is a catheter 10 having a distal end portion including an expandable element in the form of a single balloon 12 mounted on a catheter shaft 14. The balloon 12 includes an intermediate portion 16, or “barrel,” and end portions 18, 20. In one embodiment, the end portions 18, 20 of the balloon 12 reduce in diameter or taper to join the intermediate portion 16 to the catheter shaft 14 (and thus tapered end portions 18, 20 are generally termed cones or cone portions). The balloon 12 is sealed at balloon ends on the cone portions 18, 20 to allow the inflation of the balloon 12 via one or more inflation lumens 17 extending within catheter shaft 14 and communicating with the interior of the balloon 12.

As indicated in FIG. 1A, the catheter shaft 14 further includes an elongated, tubular shaft 24 at least partially nested therein. This internal shaft 24 includes a guidewire lumen 19 that directs a guidewire 26 through the catheter 10, including along the distal end portion on which the single balloon 12 is located, and out through a tip of the catheter. As illustrated in FIG. 1, this guidewire 26 may extend through the proximal end portion of the catheter 10 and through a first port 25 of a connector or hub 27 into the guidewire lumen to achieve an “over the wire” (OTW) arrangement, but could also be provided in a “rapid exchange” (RX) configuration (in which the guidewire 26 exits a lateral opening closer to the distal end, not shown) or else is fed through the tip at a passage distally of the balloon 12 (“short” RX; not shown). A second port 29 may also be associated with catheter 10, such as by way of connector or hub 27, for introducing a fluid (e.g., saline solution, a contrast agent, or both) into the interior compartment of the balloon 12 via the inflation lumen 17.

Inflatable balloon 12 may include a balloon wall 28 forming the interior for receiving the inflation fluid. The balloon 12 may be a non-compliant balloon constructed such that the balloon wall 28 maintains its size and shape in one or more directions when the balloon is inflated, and may comprise a plurality of inelastic fibers arranged in one or more layers. The balloon 12 in such case also has a substantially pre-determined surface area that remains constant during and, after inflation, has a pre-determined length and pre-determined diameter that each, or together, remain constant during and after inflation. The balloon 12 may instead be compliant (e.g., formed of an stretchable or elastic material), meaning the addition of fluid pressure causes further expansion beyond a nominal diameter (and includes semi-compliant balloons, which have a more limited degree of elasticity but thus still allow for expansion beyond a nominal diameter). The balloon 12 can have any of a variety of diameters, ranging from 1-40 mm, such as 2.0-8.0 mm, and may have any of a variety of lengths ranging from 1-300 mm. More than one inflatable balloon may be associated with the catheter 10.

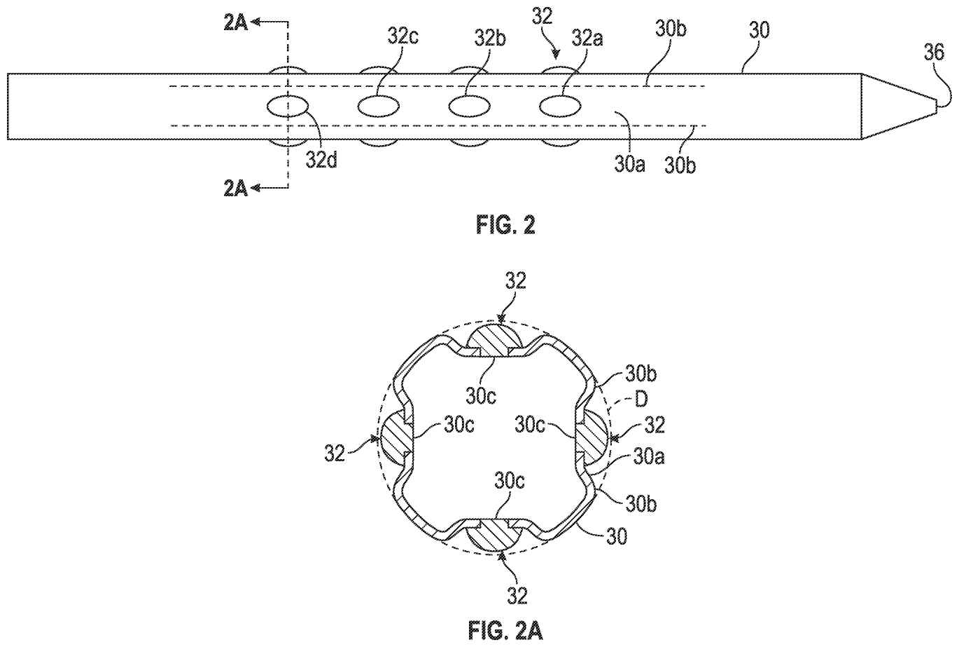

According to a first aspect of the disclosure, and with reference to FIG. 2, a universal scoring catheter is provided. This universal scoring catheter in the illustrated version comprises a tubular, elongated catheter shaft 30 adapted to provide scoring capability. This may be achieved by providing the catheter shaft 30 with one or more scoring elements 32 along an exterior thereof. In view of its tubular or hollow nature, this catheter shaft 30 may also at least partially receive the catheter 10 and, in particular, the inflatable balloon 12. Alternatively, as outlined in the description that follows, the shaft 30 may be unitary with the catheter 10, or even an extension of the catheter shaft 14 forming part of the catheter 10.

The catheter shaft 30 includes an expandable portion with the one or more scoring elements 32. This expandable portion may comprise at least one frangible portion 30a, which means a portion that may be partially, but not fully, broken away from the remainder of the shaft 30. The frangible portion(s) 30a may be formed by one or more perforations 30b, or holes, arranged on either side thereof (with adjacent frangible portions 30a thus potentially sharing the same perforations at least along one side thereof). For example, the perforations 30b may be provided in the form of generally parallel rows of holes extending longitudinally. This arrangement of perforations 30b leaves the interior portion thereof connected at proximal and distal ends to the catheter shaft 30, even once the frangible portion 30a is broken away from the remainder thereof (such as long the rows of perforations 30b).

As can be understood, the frangible portion 30a includes the one or more scoring elements 32. These scoring elements 32 may comprise one or more spaced apart beads 32a-32d (four shown, but any number greater than one may be provided) projecting from a surface of the shaft 30 and, in particular, frangible portion 30a thereof. The beads 32a-32d may also be arranged in a longitudinal row, as shown.

While only one frangible portion 30a may be provided, it is possible to provide a plurality of frangible portions 30a. For example, each frangible portion 30a may be circumferentially spaced around the catheter shaft 30, and each may include one or more scoring elements 32. In any case, the scoring element(s) 32 in total may have a length in a longitudinal direction less than a length of the frangible portion 30a, so that part of it does not include any scoring elements 32 (for example, the proximal-most and distal-most parts).

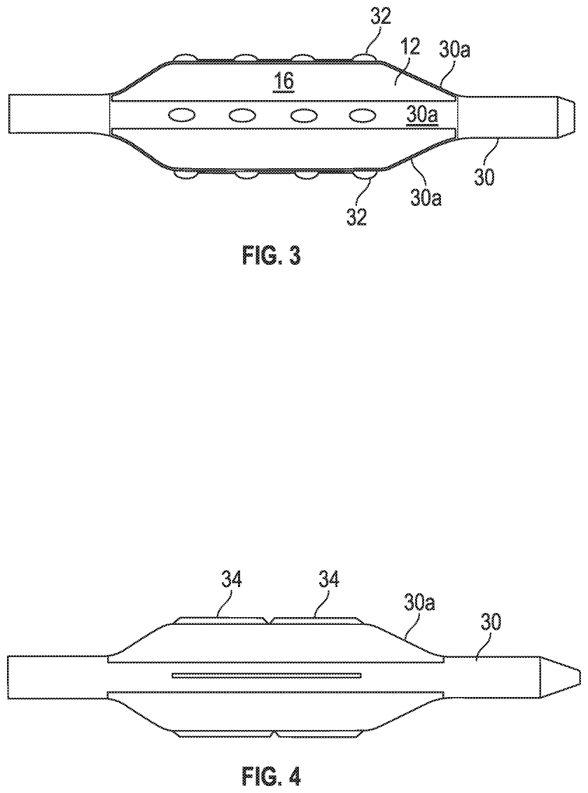

The balloon 12 in an uninflated or partially deflated condition may be positioned within the shaft 30 proximate to the frangible portion(s) 30a and the associated scoring elements 32. With reference to FIG. 3, expansion of the balloon 12 such as by way of inflation fluid may serve to break or tear the perforation(s) 30b, thereby allowing the frangible portion(s) 30a including the scoring element(s) 32 to move radially outwardly, such as for scoring a lesion, while remaining connected to the shaft 30. For this purpose, the balloon(s) 12 may have a length substantially equal to or less than the length of the frangible portion(s) 30a (such that the length of the balloon 12 from the end of the cones 18, 20 substantially matches the length of the perforations 30b and hence the length of the frangible portion(s) 30a). Continued application of internal pressure to the balloon 12 urges the scoring element(s) 32 radially outwardly into engagement with a structure, such as a lesion, associated with the vessel in which the catheter 10 is positioned.

Arrangement and positioning of the scoring element(s) 32 may correspond to the location of the intermediate or barrel portion 16 of the balloon 10, to achieve the maximum treatment effect. Deflation of the balloon 10 then allows the frangible portion(s) 30a and the associated scoring element(s) 32 to return to a retracted or non-deployed configuration, after which the catheter shaft 30 may be withdrawn or moved to another treatment area and redeployed in a similar manner.

As can be appreciated, using this arrangement allows for the creation of a universal scoring catheter using the shaft 30, in which a catheter 10 with an expandable element, such as balloon 10, may be used. The use of scoring wires is avoided, as is the need for attachment, which can reduce the tip size. Indeed, the balloon 10 or other expandable element is insulated from the scoring element(s) 32 by the presence of the intervening frangible portion(s) 30a of the shaft 30, which make contact along an interior portion with a corresponding portion of the balloon circumference.

As can be appreciated, using this arrangement allows for the scoring functionality afforded by the scoring catheter shaft 30 to be designed independently of the size of the balloon 10, since it is dependent on the arrangement of scoring element(s) 32 on the shaft 30, which may be separate from the balloon. Furthermore, different diameters of balloons 10 when inflated may be used in connection with the same catheter shaft 30 adapted for scoring, which expands the ability for use in vessels of different sizes or for different treatment regimens involving scoring. The frangible portion(s) 30a also remain fully intact until intentional force is applied by the expandable element, thus resulting in an arrangement that is unlikely to be accidentally deployed. Even once broken, the scoring elements 32 are retained by the shaft 32 as a result of the remaining connection with the frangible portion 30a and the remainder of the shaft 30.

Turning back to FIG. 2A, it can also be understood that the shaft 30 may be arranged such that the scoring element(s) 32 are recessed relative to a nominal diameter D of the shaft 30 in a retracted position. This may be achieved by forming the frangible portion 30a as a radially depressed portion of the catheter shaft 30, such as by heating and deforming during the manufacturing process. This arrangement provides a lower profile arrangement by which the scoring elements 32 in a non-deployed configuration may be tracked through a vessel (or sheath) without interference, and then deployed via expansion once the desired treatment area is reached.

Turning to FIG. 4, it can be understood that the scoring element(s) 32 may take other forms, such as one or more blades or cutters 34, which may have a straight or serrated edge. In the illustrated version, two cutters 34 are provided on each frangible portion 30a of the shaft 30. However, different arrangements of scoring element(s) 32 may be used without limitation. The scoring element(s) 32 may also vary in height above a corresponding surface of the associated frangible portion 30a of the shaft 30.

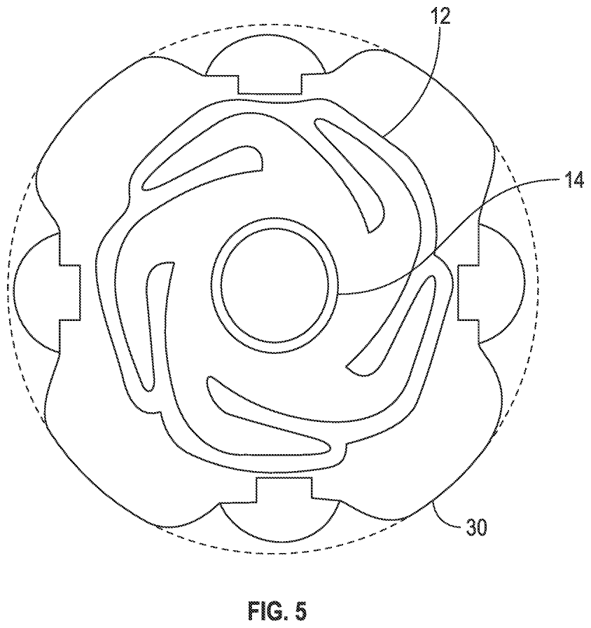

As noted previously, the catheter shaft 30 may be adapted to receiving a catheter 10 including an expandable element, such as balloon 10. Alternatively, the balloon 10 or other expandable element may be integral with the shaft 30, which may thus be an extension of shaft 14 of catheter 10 as shown in FIG. 1. In this situation, the shaft 30 would thus include a shaft 14, as shown in the cross-section of FIG. 5 (with the expandable element or balloon 12 shown in a folded condition). In other words, the position of the balloon 10 may be fixed within scoring catheter shaft 30 and attached to it, or the balloon 10 and associated shaft(s) 14 may be detached from the shaft 30 and fully insertable into it and removable from it.

The perforations 30b may be formed using a cutter, such as a laser, for forming holes or slots in the shaft 30 alternating with uninterrupted material. The arrangement provided is such that the outward pressure afforded by the balloon 10 to break the connection(s) and allow for the frangible portion(s) 30a to deploy. While a number of perforations 30b are shown, it can be appreciated that only a single pair of spaced perforations on either side of the scoring elements 32 may be used to form the frangible portion 30a.

As indicated in FIG. 2A, the scoring element(s) 32 may take the form of rivets, which project partially into holes 30c formed in the shaft 30. These holes 30c may be formed using a cutter, such as a laser. The rivets for forming the scoring element(s) 32 may then be press-fitted into the holes 30c. These rivets may be formed of metal or plastic, and heat staking may be used to form a locking flange via a heated die. Any combinations of materials, sizes, shapes, or structures may be used.

The shaft 30 may comprise a polymer material, such as for example Pebax, polyurethane, polyethylene terephthalate (PET), thermoplastic elastomers, PEEK, or the like. The perforations 30b may be provided such that inflation of the balloon 12 to a nominal diameter is sufficient to break away the frangible portion 30a from the remainder of the shaft 30. The scoring elements 32 may comprise a polymer, such as a hard plastic, or metal. Radiopaque materials may also be used as necessary or desired to aid in identifying the catheter 10 or shaft 30 (including possibly the scoring elements 32) under fluoroscopy.

Summarizing, this disclosure relates to the following items:
1. An apparatus for scoring a lesion in a vessel, comprising: a catheter shaft comprising a frangible portion including a scoring element adapted for advancing radially outwardly for scoring the lesion when the frangible portion is broken.
2. The apparatus of item 1, wherein the frangible portion comprises perforations arranged on either side of the scoring element.
3. The apparatus of item 1 or item 2, wherein the scoring element comprises one or more beads, blades, serrations, or a combination thereof.
4. The apparatus of any of items 1-3, wherein the catheter shaft comprises a plurality of frangible portions circumferentially spaced around the catheter shaft, each associated with a scoring element.
5. The apparatus of item 4, wherein each frangible portion comprises longitudinal perforations arranged on either side of the scoring element.
6. The apparatus of any of items 1-5, wherein the scoring element has a length in a longitudinal direction equal to or less than a length of the frangible portion.

7. The apparatus of any of items 1-6, wherein the frangible portion comprises a radially depressed portion of the catheter shaft.
8. The apparatus of any of items 1-7, further including an expandable element within the catheter shaft for breaking the frangible portion.
9. The apparatus of item 8, wherein the expandable element comprises a balloon.
10. An apparatus for scoring a lesion in a vessel, comprising:
   a catheter shaft including an expandable portion adapted for scoring the lesion, the catheter shaft including a plurality of perforations forming the expandable portion.
11. The apparatus of item 10, wherein the expandable portion of the catheter shaft includes a scoring element, and the plurality of perforations comprise perforations located on either side of the scoring element.
12. The apparatus of item 11, wherein the scoring element comprises one or more beads, blades, serrations, or a combination thereof.
13. The apparatus of item 11 or item 12, wherein the expandable portion comprises a depressed portion of the catheter shaft.
14. The apparatus of any of items 11-13, wherein catheter shaft includes a plurality of scoring elements, and the plurality of perforations comprise perforations located on either side of the depressed portion.
15. The apparatus of any of items 10-14, further including a balloon catheter adapted to be inserted into the catheter shaft, whereby expansion of the balloon serves to partially break the expandable portion from the catheter shaft.
16. The apparatus of item 15, wherein the balloon has a length equal to or less than a length of the expandable portion.
17. An apparatus for scoring a lesion in a vessel, comprising:
   a catheter shaft including a depressed portion having a scoring element adapted for scoring the lesion when the depressed portion is urged radially outwardly to a deployed position.
18. The apparatus of item 17, wherein the depressed portion further comprises a frangible portion of the catheter shaft.
19. The apparatus of item 17 or item 18, further including an expandable element within the catheter shaft for breaking the frangible portion.
20. The apparatus of item 19, wherein the expandable element comprises a balloon catheter.
21. A method for scoring a lesion in a vessel in connection with an expandable element, comprising:
   breaking apart a catheter shaft to cause a scoring portion thereon to assume a deployed position for scoring the lesion.
22. The method of item 21, wherein the breaking apart step comprises expanding an expandable balloon within the catheter shaft.
23. The method of item 21 or item 22, wherein the breaking apart step comprises tearing perforations formed in the catheter shaft on either side of a scoring element.
24. A method for scoring a lesion in a vessel in connection with an expandable element, comprising:
   expanding a portion of catheter shaft from a depressed position to cause a scoring element thereon to assume a deployed position for scoring the lesion.
25. The method of item 24, wherein the expanding step comprises breaking a frangible portion of the catheter.
26. The method of item 24, wherein the breaking step comprises tearing perforations formed in the catheter shaft on either side of the scoring element.

As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed.
“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. An apparatus for scoring a lesion in a vessel, comprising:
a catheter shaft comprising a frangible portion including a scoring element adapted for advancing radially outwardly for scoring the lesion when the frangible portion is broken.

2. The apparatus of claim 1, wherein the frangible portion comprises perforations arranged on either side of the scoring element.

3. The apparatus of claim 1, wherein the scoring element comprises one or more beads, blades, serrations, or a combination thereof.

4. The apparatus of claim 1, wherein the catheter shaft comprises a plurality of frangible portions circumferentially spaced around the catheter shaft, each associated with a scoring element.

5. The apparatus of claim 4, wherein each frangible portion comprises longitudinal perforations arranged on either side of the scoring element.

6. The apparatus of claim 1, wherein the scoring element has a length in a longitudinal direction equal to or less than a length of the frangible portion.

7. The apparatus of claim 1, wherein the frangible portion comprises a radially depressed portion of the catheter shaft.

8. The apparatus of claim 1, further including an expandable element within the catheter shaft for breaking the frangible portion.

9. The apparatus of claim 8, wherein the expandable element comprises a balloon.

10. An apparatus for scoring a lesion in a vessel, comprising:
a catheter shaft including an expandable portion adapted for scoring the lesion, the catheter shaft including a plurality of perforations forming the expandable portion.

11. The apparatus of claim 10, wherein the expandable portion of the catheter shaft includes a scoring element, and the plurality of perforations comprise perforations located on either side of the scoring element.

12. The apparatus of claim 11, wherein the scoring element comprises one or more beads, blades, serrations, or a combination thereof.

13. The apparatus of claim 11, wherein the expandable portion comprises a depressed portion of the catheter shaft.

14. The apparatus of claim 11, wherein catheter shaft includes a plurality of scoring elements, and the plurality of perforations comprise perforations located on either side of the depressed portion.

15. The apparatus of claim 10, further including a balloon catheter adapted to be inserted into the catheter shaft, whereby expansion of the balloon serves to partially break the expandable portion from the catheter shaft.

16. The apparatus of claim 15, wherein the balloon has a length equal to or less than a length of the expandable portion.

17. An apparatus for scoring a lesion in a vessel, comprising:
a catheter shaft including a depressed portion having a scoring element adapted for scoring the lesion when the depressed portion is urged radially outwardly to a deployed position.

18. The apparatus of claim 17, wherein the depressed portion further comprises a frangible portion of the catheter shaft.

19. The apparatus of claim 17, further including an expandable element within the shaft for breaking the frangible portion.

20. The apparatus of claim 19, wherein the expandable element comprises a balloon catheter.

21-26. (canceled)
